(19) United States
(12) Patent Application Publication
(10) Pub. No.: US 2014/0275292 A1
(43) Pub. Date: Sep. 18, 2014

(54) SYSTEMS AND METHODS EMPLOYING HUMAN STEM CELL MARKERS FOR DETECTION, DIAGNOSIS AND TREATMENT OF CIRCULATING TUMOR CELLS

(71) Applicant: WILLIAM MICHAEL SCHOPPERLE, South Boston, MA (US)

(72) Inventor: WILLIAM MICHAEL SCHOPPERLE, South Boston, MA (US)

(21) Appl. No.: 14/206,622

(22) Filed: Mar. 12, 2014

Related U.S. Application Data
(60) Provisional application No. 61/935,565, filed on Feb. 4, 2014.
(60) Provisional application No. 61/781,961, filed on Mar. 14, 2013.

(51) Int. Cl. G01N 33/574

(52) U.S. Cl. CPC G01N 33/57492
USPC 514/789; 435/7.23; 435/287.2

(57) ABSTRACT

The invention relates to systems and methods to detect circulating tumor cells in a blood sample obtained from a cancer patient. Further, this invention relates to systems and methods to determine a diagnosis or prognosis of aggressive and metastatic cancer by determining the presence and/or level of circulating tumor cells in a blood sample obtained from a cancer patient. The detection and diagnosis is based on the presence or absence of cells that express podocalyxin and/or TRA biomarkers.

### CROSS REFERENCE TO RELATED PATENT APPLICATIONS

This patent application claims the benefit of priority under 35 U.S.C. §119(e) from U.S. Provisional Patent Application No. 61/935,565 entitled “Systems and Methods Employing Human Stem Cell Markers for Detection, Diagnosis and Treatment of Circulating Tumor Cells”, filed on Feb. 4, 2014; and U.S. Provisional Patent Application No. 61/781,961 entitled “Systems and Methods Employing Human Stem Cell Markers for Detection, Diagnosis and Treatment of Circulating Tumor Cells”, filed on Mar. 14, 2013; which are incorporated in their entirety herein by reference.

### FIELD OF THE INVENTION

This invention relates to systems and methods to detect circulating tumor cells in a blood sample obtained from a cancer patient. Further, this invention relates to systems and methods to determine a diagnosis or prognosis of aggressive and metastatic cancer by determining the presence and/or level of circulating tumor cells in a blood sample obtained from a cancer patient.

### BACKGROUND OF THE INVENTION

Most solid-tumor human cancers can be broadly categorized into two general types, localized cancer and aggressive/metastatic cancer. Localized cancer does not spread, is generally non-lethal, and is usually manageable and treatable with current standard cancer therapies. In contrast, aggressive cancer generally spreads to other parts of the body, usually is not curable with conventional cancer therapies, and is almost always lethal for the patient. One of the hypotheses of the stem cell theory of cancer is that aggressive and metastatic cancer is caused by a unique type of cancer cells which have stem cell-like properties. These unique cancer cells are derived from normal adult stem cells which reside within the body. Another hypothesis is that localized cancer cells transform into aggressive cancer cells through the acquisition of specialized stem-cell functions allowing them to detach from the primary tumor, move through surrounding tissues and into the blood supply and finally, to reattach and seed new tumor growth elsewhere in the body.

It is believed that the cancer cells having stem cell-like properties make up only a small portion of the tumor bulk, but it is further believed that are the cells which are highly resistant to current therapies, are highly motile, can re-seed the tumor and can cause new growth in other parts of the body. Moreover, it is believed that these cancer stem cells are responsible for driving the metastatic/aggressive cancer and these cancer stem cells have an embryonic or pluripotent genomic signature.

There are many patients that are diagnosed with localized cancer tumors. The cancer tumors may be detected based on the presence of a mass in a patient. The diagnosis of cancer based on the presence of the mass, i.e., the tumor itself, may result in a late stage diagnosis. Further, cancer diagnosis based on the presence of the mass may not provide information as to whether the cancer cells are localized or aggressive, i.e., a metastatic stage.

It is often difficult to identify and diagnose cancer at a pre-invasive stage because the carcinoma can be asymptomatic. The diagnosis is typically provided following a surgical biopsy and this diagnostic procedure is generally only carried out in patients at-risk or following a clinical examination and/or ultrasonography with a suspicious outcome. The diagnostic procedures currently available include surgical biopsy which is most often performed as an open incision of a tissue sample and sometimes as a needle-biopsy. After proper histological processing, the sample is examined by a pathologist. The finding of cancer cells will typically be treated by irradiation and sometimes with adjuvant chemotherapy.

Known diagnostic procedures can be expensive, time-consuming and invasive. Further, these known procedures may not be capable of determining if the cancer that is detected is of an aggressive form.

A small percentage, for example, less than 10%, of localized prostate cancer tumors will develop into an aggressive, metastatic stage. Known cancer treatments can be less effective or non-effective against aggressive cancer cells and therefore, these cancer cells can survive cancer treatments and can re-seed the cancer causing new tumor growth following treatment. It would be beneficial to know which patients will develop the aggressive disease and which patients' disease will not proceed beyond the localized stage. Thus, those patients that are known to develop the aggressive disease can receive early treatment and those that will not, can conclude their treatment. It has, however, proven to be difficult to determine which patients will develop the aggressive disease.

The time course for progression from localized prostate cancer to lethal aggressive/metastatic cancer has significant variability. Some cancers progress rapidly while others remain indolent for years prior to progressing to an aggressive/metastatic form, and some cancers will never progress to an aggressive/metastatic form. It is not known why some cancers progress rapidly to an aggressive/metastatic form while others do not. For example, annually in the United States, approximately 240,000 men will be diagnosed with localized prostate cancer and studies have estimated that only about 10% of the hundreds of thousands of men diagnosed with localized prostate cancer will actually develop the lethal aggressive/metastatic form of the disease.

The standard protocol for diagnosing localized cancer is a biopsy. Several tissue cores are harvested from the cancer tissue and are histologically examined by a pathologist. If abnormal looking cells are identified based on cell morphology, an assessment is made as to how undifferentiated the prostate cells look as compared with normal cells. In certain embodiments, such as for prostate cancer, the assessment is made based on Gleason score. The Gleason score is a histological test which was developed about sixty years ago having an original score range from 2 to 10 based on cell morphology. Today, for the most part, in actual clinical practice, the Gleason score ranges from Gleason 6 which is the least undifferentiated tissue as compared to normal tissue (this is diagnosed as the lowest grade/least aggressive prostate cancer) to Gleason 10 which is the most undifferentiated prostate tissue (this is diagnosed as the highest grade/most aggressive prostate cancer). In general, the higher the Gleason score, the more likely the patient will develop the aggressive/metastatic form of prostate cancer. However, the Gleason score is not accurate for all cancer patients. For example, it has been demonstrated that patients with Gleason 6 localized prostate cancer will develop the aggressive disease while patients with Gleason 10 localized prostate cancer will not progress beyond localized cancer.

Typical treatment for patients diagnosed with cancer, such as prostate cancer, is surgery or radiation. However, given the small percentage of patients who eventually develop the aggressive form of cancer, and the serious complications associated with cancer therapies, there is growing a concern that many cases of prostate cancer are incorrectly diagnosed and most patients are undergoing treatments that are not necessary. In fact many new clinical studies have revealed that the current test for diagnosing prostate cancer (Gleason score and PSA blood test) has little effect on the annual death rate from prostate cancer as compared with no screening at all.

There is a tremendous need for new and better diagnostic and prognostic clinical tests to determine which patients have aggressive and deadly cancer and therefore should be treated, and which patients have only localized and non-threatening cancer (or no cancer at all) and therefore should not be treated but rather actively monitored for any biological changes within the localized cancer tissue. By placing selected patients on active surveillance, the number of unnecessary cancer treatments received by cancer patients having localized cancer that does not develop into aggressive cancer, may be reduced. However, there is a concern as to how to select those patients with local cancer that should be placed on active surveillance and those that should receive cancer treatment. The concern is due at least in part to the current clinical protocols employed to diagnose cancer and the limitations associated therewith for identifying which patients with local cancer will progress to aggressive cancer and which patients with local cancer will not progress to aggressive cancer.

An emerging technology in cancer detection relates to the identification of circulating tumor cells in peripheral whole blood of a cancer patient. Studies have suggested that the presence of circulating tumor cells in peripheral blood is associated with higher forms or aggressive and metastatic cancer, and decreased overall survival rates of cancer patients. In accordance with this technology, a peripheral whole blood sample with a high level of circulating tumor cells present is indicative of aggressive cancer and a peripheral whole blood sample with a low level or absence of circulating tumor cells is indicative of localized cancer. Further, in developing a treatment regimen, a patient having a low level or absence of circulating tumor cells may be placed on an active surveillance program and a patient having a high level of circulating tumor cells may receive treatment for aggressive cancer.

Several diagnostic circulating tumor cell test assays have been developed to identify circulating tumor cells of epithelial origin. These assays employ markers, such as CD45, EpCAM and Cytokeratins 8, 18 and 19. There are disadvantages associated with these markers and their use in diagnosing aggressive cancer in a patient. For example, there has been limited success in identifying epithelial-like circulating tumor cells in blood and there is uncertainty as to whether the circulating tumor cells detected in peripheral whole blood actually originate from the primary tumor. In general, the tests conducted using the known markers (e.g., CD45, EpCAM and Cytokeratins 8, 18 and 19) have not been shown to accurately predict whether the cancer is an aggressive or a non-aggressive form.

Thus, there is a need in the art to identify biomarkers which effectively and accurately detect and isolate circulating tumor cells in the peripheral whole blood of a cancer patient in order to improve diagnostic testing and treatment procedures for patients with aggressive cancer.

The detection or identification of podocalyxin on certain cancer cells within localized cancer or within tissue may be used to identify aggressive cancer stem cells or aggressive cancer cells with stem cell-like properties.

Podocalyxin is a cell surface plasma membrane protein which is highly expressed on the surface of human pluripotent stem cells, such as human embryonic stem cells, and is a human stem cell-defining marker for human embryonic stem cells. Pluripotent stem cells are fundamental stem cells having biological properties to differentiate into any one of the two hundred different kinds of cells that make up the human body. Podocalyxin has several putative cellular functions which include a cellular adhesion function, an anti-adhesion protein and a cell motility function. Podocalyxin can be cleaved or clipped off the surface of the cell by a biological process to generate a soluble form that can enter the blood of a patient. Thus, although the exact functions of podocalyxin in cancer have yet to be determined, these functions of podocalyxin expressed on cancer stem cells may allow the cancer cells to survive within a patient and furthermore, to spread or metastasize and also re-seed the tumor after standard cancer therapy treatments.

TRA (also referred to as an epitope) is a carbohydrate (sugar) molecular structure which is present in very large amounts on the cell surface of undifferentiated human embryonic pluripotent stem cells. This carbohydrate molecule was first discovered several decades ago on human embryonal carcinoma pluripotent cancer stem cells. The TRA carbohydrate molecule was discovered on embryonal carcinoma by using two distinct mouse monoclonal antibodies named TRA-1-60 and TRA-1-81. At the time of discovery, the epitopes which these monoclonal antibodies bind to was unknown and therefore, the epitopes where named after the antibodies TRA-1-60 and TRA-1-81. It is now believed that the TRA carbohydrate structure is a pluripotency associated type 1 lactosamine epitope and the minimal epitope recognized by the TRA-1-60 and TRA-1-81 antibodies is Galβ1-3G1cNAcβ1-3Galβ1-4GlcNAc on an extended tetrasaccharide mucin type O-glycan structure in human embryonic stem cells with a likely structure Galβ1-3GlcNAcβ1-3Galβ1-4GlcNAcβ1-6(Ga1β1-3)GalNAc, which is present in human embryonic stem cells and cancer stem cells as part of a mucin type 0-glycan structure. An epitope is the molecular structure which a particular antibody specifically reacts with and attaches or binds to. The TRA-1-60 and TRA-1-81 epitopes are human biomarkers for pluripotent embryonic stem cells. All pluripotent stem cells, regardless of how they are generated, have the TRA-1-60/TRA-1-81 carbohydrate structure on the surface of their cells.

Podocalyxin is a ‘molecular carrier’ of TRA molecular structures expressed on human embryonal carcinoma stem cells. Purified podocalyxin has binding activity with TRA molecular structures through cell post-translational modification mechanisms to form a podocalyxin/TRA molecule on the surface of cancer stem cell or cancer cells having stem cell-like properties. Since podocalyxin can be cleaved or clipped-off the surface of the cell and enter the blood of a patient and TRA molecules may be attached to podocalyxin, the TRA molecules can also detach from the cell and enter the blood of the patient. TRA may also be released into the blood without being attached to podocalyxin and furthermore, TRA may be attached to cell surface molecules other than podocalyxin.

It is desirous to develop systems and methods for detecting the presence of circulating tumor cells in a blood sample of a patient based on the presence or absence of cells that express podocalyxin and/or TRA and providing a diagnosis and prognosis therefrom. Further, systems and methods of the invention include selecting and implementing an effective therapy and treatment regimen for the patient.

### SUMMARY OF THE INVENTION

In one aspect, this invention provides a method of detecting the presence of circulating tumor cells in a patient. The method includes obtaining a blood sample from the patient, testing the sample to determine a presence of podocalyxin and/or TRA molecules; and determining the presence or absence of circulating tumor cells based on the presence or absence of podocalyxin and/or TRA molecules in the sample. Further, the method can include providing a medical diagnosis of the presence of aggressive or metastatic cancer in the patient based on the presence of the circulating tumor cells in the sample, and providing a medical diagnosis of the absence of aggressive or metastatic cancer in the patient based on the absence of the circulating tumor cells in the sample.

In another aspect, the invention provides a device for detecting circulating tumor cells diagnosing aggressive cancer in a patient. The device includes a tool for obtaining a blood sample from the patient. The tool can be a syringe. The device further includes a detector means for determining a presence or an absence of podocalyxin and/or TRA molecules in the blood sample. Furthermore, the device includes an indicator means to display the presence or absence of the podocalyxin and/or TRA molecules in the blood sample.

Furthermore, the invention provides a kit for testing for circulating tumor cells and for aggressive or metastatic cancer in a blood sample obtained from a patient.

In another aspect, this invention provides a method of selecting a medical treatment for a patient having cancer. The method includes obtaining a blood sample from the patient, evaluating the sample for a presence of circulating tumor cells, determining the presence or absence of podocalyxin and/or TRA in the blood sample, determining the presence of circulating tumor cells based on the presence or absence of podocalyxin and/or TRA in the blood sample, providing a positive medical diagnosis or prognosis of aggressive cancer for the patient based on the presence of the circulating tumor cells in the blood sample; providing a negative medical diagnosis or prognosis of aggressive cancer for the patient based on the absence of circulating tumor cells in the blood sample, administering a treatment regimen selected from radiation, chemotherapy, surgery and a combination thereof, for the positive medical diagnosis or prognosis of aggressive cancer, and selecting an active surveillance and monitoring treatment regimen for the negative medical diagnosis or prognosis of aggressive cancer.

In still another aspect, this invention provides a method of measuring a level of podocalyxin and/or TRA in a blood sample of a patient to determine the level of circulating tumor cells in the blood sample. The method includes obtaining a blood sample from the localized tumor and measuring the level of podocalyxin and/or TRA in the sample.

Further, the method can include comparing the level of podocalyxin and/or TRA in a blood sample obtained from a patient to a control sample obtained from a non-cancer-containing patient or a biological sample earlier obtained from the patient. If the level of podocalyxin and/or TRA in the blood sample is measurably higher than the level of podocalyxin and/or TRA in the control sample, then there is a diagnosis of the presence of circulating tumor cells in the patient. If the level of podocalyxin and/or TRA in the blood sample is the same or less than the level of podocalyxin and/or TRA in the control sample, then there is a diagnosis of the absence of circulating tumor cells in the patient.

Furthermore, the method can include comparing the level of podocalyxin and/or TRA in a blood sample obtained from a patient to a threshold podocalyxin and/or TRA level or threshold podocalyxin and/or TRA range. If the level of podocalyxin and/or TRA in the blood sample is measurably higher than the threshold podocalyxin and/or TRA level or range, then there is a diagnosis of the presence of circulating tumor cells in the patient. If the level of podocalyxin and/or TRA in the blood sample is the same or less than the threshold podocalyxin and/or TRA level or range, then there is a diagnosis of the absence of circulating tumor cells in the patient.

In certain embodiments, the cancer is prostate cancer.

### DETAILED DESCRIPTION OF THE INVENTION

This invention includes a device and method for detecting the presence of circulating tumor cells in the peripheral whole blood of a cancer patient. Further, this invention relates to biomarkers to detect, diagnose, measure and monitor circulating tumor cells in the patient. Furthermore, this invention includes a device and method for evaluating a blood sample obtained from a cancer patient to determine the presence or absence of circulating tumor cells based on the presence or absence of podocalyxin, TRA and combinations or mixtures thereof, in the blood sample. Furthermore, this invention includes a device and method for detecting and diagnosing aggressive cancer in a patient based on the presence or absence of circulating tumor cells in the blood sample of the patient. The invention employs podocalyxin, TRA and combinations or mixtures thereof as a prognostic marker in a blood sample obtained from a cancer patient, such that the presence of podocalyxin and/or TRA is indicative of the presence of circulating tumor cells in the blood sample which indicates a prognosis or diagnosis of aggressive or metastatic cancer. Moreover, this invention includes a device and method for providing a therapy and treatment regimen for a patient based on the presence or absence of circulating tumor cells in the blood sample of the patient. This invention also includes a test kit.

Aggressive or metastatic cancer is caused by stem cell-like cancer cells. Aggressive cancer cells are highly resistant and motile. Further, aggressive cancer cells can survive conventional cancer treatments. Without detection, diagnosis and treatment, aggressive and resistant cancer cells may grow and metastasize throughout the body of the patient and potentially, result in a lethal form of cancer.

As used herein and the claims, the terminology “podocalyxin-expressing cells” and related variations thereof, means a cancer stem cell with the podocalyxin protein molecule present on the surface of the cancer stem cell, or podocalyxin could be present intracellularly in the cytoplasm or associated with an organelle, or podocalyxin could be present in the extracellular space and/or the glycocalyx surrounding the cancer stem cell and not necessarily in direct physical contact with the cancer stem cell.

As used herein and the claims, the terminology “TRA-expressing cells” and related variations thereof, means a cancer stem cell with the TRA molecule present on the surface of the cancer stem cell, or TRA could be present intracellularly in the cytoplasm or associated with an organelle, or TRA could be present in the extracellular space and/or glycocalyx surrounding the cancer stem cell and not necessarily in direct physical contact with the cancer stem cell.

Further, as used herein and the claims, the terminology “cancer stem cells” and related variations thereof, means a cancer stem cell with the ability to differentiate into other cell types, or a cancer cell with some properties which are shared with stem cells but the cell may not be able to differentiate into other cell types, or a cell which is not defined as cancer by current medical standards but would have cellular changes which would not be present in a normal cell. Moreover, for ease of description, the invention is described herein with respect to prostate cancer. However, this description is not intended to be limiting. It is contemplated and should be understood that the invention relates to all types of cancer tumors, and cancer stem cells.

Podocalyxin is a cell surface plasma membrane protein which is expressed in a few types of normal cells and is highly expressed on the surface of human pluripotent stem cells (e.g., cancer cells). As used herein, the term “normal” refers to the absence of cancer. Pluripotent stem cells are fundamental stem cells having biological properties to differentiate into any one of the two hundred different kinds of cells that make up the human body.

The TRA molecule is an embryonic and pluripotent stem cell specific structure. TRA is present on normal stem cells, such as embryonic stem cells and the like, and cancer stem cells.

These podocalyxin-expressing and/or TRA-expressing cancer stem cells make up only a small portion of the tumor bulk, but they have stem cell-like properties. The podocalyxin-expressing and/or TRA-expressing cancer stem cells can migrate or move out of the tumor bulk and enter the peripheral whole blood of the patient. These cancer stem cells which are present in the peripheral whole blood are referred to as circulating tumor cells. The circulating tumor cells are highly resistant and motile, can re-seed the tumor and cause new growth elsewhere in the body. Podocalyxin and TRA can bind together. TRA may also attach or bind to another molecule, e.g., other than podocalyxin, on the migrating tumor cell. Therefore, the cancer stem cells which migrate or move out from the tumor bulk, i.e., circulating tumor cells, and enter the peripheral whole blood can have podocalyxin and/or TRA expressed on the surface. A test to determine if there are any circulating tumor cells in a blood sample of a patient can include using podocalyxin-specific antibodies (e.g., or any other polyclonal or monoclonal antibodies specific to the podocalyxin structure) or TRA-specific antibodies to detect the presence of any podocalyxin-expressing and/or TRA-expressing cancer stem cells. If podocalyxin and/or TRA reactivity is positive, this indicates that there are circulating tumor cells in the blood which will eventually lead to the aggressive/metastatic form of the cancer. Thus, the patient is administered aggressive treatment. If no positive podocalyxin and/or TRA reactivity is detected, this indicates that there are little or no circulating tumor cells present in the blood and the cancer is not aggressive and no aggressive treatment is needed for the patient. This test can be particularly useful for determining the treatment for prostate cancer patients with localized disease by identifying circulating tumor cells with podocalyxin and/or TRA antibodies and assessing the aggressiveness of a localized tumor.

It is contemplated that cells in blood expressing podocalyxin and/or TRA may be present in normal patients at zero or low levels and may be present in patients having aggressive cancers at higher levels. In accordance with the invention, in certain embodiments, the number of cells expressing podocalyxin and/or TRA in a blood sample obtained from a cancer-containing patient can be compared to the number of cells expressing podocalyxin and/or TRA in a control sample obtained from a non-cancer-containing patient. If the number of cells expressing podocalyxin and/or TRA in the blood sample from the cancer-containing patient is measurably higher than the number of cells expressing podocalyxin and/or TRA in the control sample, then there is a diagnosis of the presence of circulating tumor cells in the sample. In contrast, if the number of cells expressing podocalyxin and/or TRA in the blood sample from the cancer-containing patient is less than or essentially equal to the number of cells expressing podocalyxin and/or TRA in the control sample, then there is a diagnosis of the absence of circulating tumor cells in the sample.

In certain other embodiments, the number of cells expressing podocalyxin and/or TRA in a blood sample obtained from a cancer-containing patient can be compared to the number of cells expressing podocalyxin and/or TRA in another, e.g., earlier obtained, blood sample from the cancer-containing patient. If the number of cells podocalyxin and/or TRA in the blood sample from the cancer-containing patient is measurably higher than the number of cells expressing podocalyxin and/or TRA in the other, e.g., earlier obtained, blood sample from the cancer-containing patient, then there is a diagnosis of the presence of circulating tumor cells in the patient. In contrast, if the number of cells expressing podocalyxin and/or TRA in the blood sample of the cancer-containing patient is less than or essentially equal to the number of cells expressing podocalyxin and/or TRA in the other, e.g., earlier obtained, blood sample from the cancer-containing patient, then there is a diagnosis of the absence of circulating tumor cells in the patient.

In other embodiments, the presence or absence of circulating tumor cells may be determined based on comparison of the number of cells expressing podocalyxin and/or TRA in the blood sample to a threshold value. There may be a threshold level of the number of cells expressing podocalyxin and/or TRA such that when the number of cells expressing podocalyxin and/or TRA measured in the blood sample is above or essentially equal to the threshold number, the presence of circulating tumor cells is determined and when the number of cells expressing podocalyxin and/or TRA measured in the blood sample is below the threshold number, the absence of circulating tumor cells is determined.

In accordance with the invention, in certain embodiments, a threshold number of cells expressing podocalyxin and/or TRA or a threshold range of the number of cells expressing podocalyxin and/or TRA found in normal, healthy patients may be determined by obtaining blood samples from these patients and measuring the number of cells expressing podocalyxin and/or TRA in these patients. Based on this data, a threshold number or threshold range can be established and employed to determine the presence or absence of circulating tumor cells in blood samples. When the number cells expressing podocalyxin and/or TRA in a blood sample from a cancer-containing patient is measurably above or essentially equal to the threshold number or threshold range of cells expressing podocalyxin and/or TRA, a diagnosis of the presence of circulating tumor cells is made. When the number of cells expressing podocalyxin and/or TRA in a blood sample from a cancer-containing patient is below the threshold number or range of cells expressing podocalyxin and/or TRA, a diagnosis of the absence of circulating tumor cells is made.

The advantage of the systems and methods of this invention over those currently employed for the detection of cancer can include early detection or prognosis of aggressive cancer, and the ability to customize a treatment regimen in accordance with the aggressive nature of the cancer. Since the blood sample is easily collected, tested and evaluated, aggressive cells can be detected in a timely manner using a non-invasive procedure. Further, it is not necessary to remove tissue or cells for biopsy and surgery. The analysis of the blood sample is performed in a specific manner to reduce the risk of obtaining false positive and false negative results.

Further, the systems and methods of this invention allow for an early detection or prognosis of a lack of aggressive cancer progression based on the absence of circulating tumor cells in the blood, such that a treatment regimen can be customized for the patient. For example, the patient can be placed on an aggressive treatment regimen if a presence of podocalyxin and/or TRA expressing cells is detected and there is a prognosis of aggressive cancer progression. Suitable and conventional examples of aggressive treatment regimens include radiation, chemotherapy, surgery and combinations thereof. Further, for example, the patient can be merely placed a non-aggressive treatment regimen if an absence of cells in blood that express podocalyxin and/or TRA is detected and there is a prognosis of a lack of aggressive cancer progression. Suitable and conventional examples of non-aggressive treatment regimens include active surveillance and monitoring, without the need to be subjected to radiation, chemotherapy, surgery, or combinations thereof Thus, customizing treatment regimens based on the results can be effective in reducing or precluding unnecessary therapies and treatments for a cancer-containing patient.

The blood sample can be collected from the patient using a variety of conventional tools known in the art, such as using a syringe or like device, and conventional methods. The blood sample can be collected in a labeled container or placed in a labeled container. The label of the container may contain a unique identification number and one part of the label may be transferable to a slide. The sample can be collected in a container which may contain stabilizing agents to aid in preservation of the sample and of the signal/marker quality in the sample. Stabilizing agents include but are not limited to pH-buffers, Protease-inhibitors, RNase inhibitors, fixatives and other compounds/components known to persons skilled in the art. Collected samples may be stored at the site of collection at suitable temperature or they may be transported to a local or external laboratory for preparation.

The sample may be processed using various conventional techniques in order to obtain an optimal signal from any circulating tumor cells present in the sample. Processing the sample may include, but is not limited to, filtration, precipitation, immunoprecipitation, flow-sorting, lyzing, centrifugation, cooling, freezing, heating or any other methods known to a person skilled in the art. Preferably, the sample is treated to allow optimal detection of cells that have podocalyxin and/or TRA molecules expressed on the surface the cell. This is, for example, performed by treating the sample in a manner that allows the molecules/cells of the sample to remain intact and, as far as possible, also retain their original morphology.

The sample may be analyzed for the presence of podocalyxin and/or TRA biomarkers in order to detect circulating tumor cells and aggressive cancer cells using a variety of analyses. These analyses include immunoassays, immunostaining, immunofluorescence, immunohistochemistry, direct IHC, indirect IHC, immunocytochemistry, is situ hybridization, fluorescent ISH, FISH in suspension, western blot, flow cytometry, fluorescence-activated cell sorting, imageStream, turtle probes, target primed rolling circle PRINS, luminex assay, polymerase chain reaction, mass spectrometry and the like.

In certain embodiments, analyzing the blood samples includes the use of a binding material which is selected to interact with podocalyxin and/or TRA molecules. The binding material may be effective to separate and isolate the podocalyxin and/or TRA biomarkers and/or circulating tumor cells from the blood sample. Suitable binding materials are known in the art or may be prepared. In accordance with certain embodiments, the binding material is an antibody.

As above-identified, this description is directed to prostate cancer. However, it is contemplated that the systems and methods described herein for prostate cancer are equally applicable to tumors in other organs or tissues in the body. For example, and without limitation, the ovary, colon, and breast.

In demonstrating the effectiveness of this invention, it is contemplated that blood samples will be collected from patients with localized cancer and patients with metastatic cancer. The samples collected from these two groups of patients will be evaluated to determine the presence or absence of circulating tumor cells by determining the presence of absence or level of podocalyxin-expressing, TRA-expressing, and/or podocalyxin/TRA-expressing cancer stem cells. Further, it is anticipated that the levels of podocalyxin and/or TRA in the samples may be measured by standard methods (e.g., elisa assays, western blotting and the like) using podocalyxin-specific and/or TRA-specific antibodies. These antibodies are commercially available from numerous sources and are widely used by the human stem cell research community for the characterization of embryonic stem cells. The levels of circulating tumor cells in the samples of patients having cancer (localized and metastatic) can be compared with each other, and can be compared with the healthy control samples.

The podocalyxin and TRA are markers of human stem cells. As previously indicated, there may be low levels of podocalyxin and TRA in a healthy individual due to the presence of podocalyxin and TRA on normal stem cells such vascular cells. However, in a disease state, the levels of podocalyxin and TRA may be increased as compared to the levels present in the healthy/normal body.

Further, in accordance with certain embodiments of the invention, antibodies to podocalyxin and TRA can be introduced into the body of a patient having a form of aggressive cancer. These antibodies may be effective to target podocalyxin, TRA carbohydrate molecular structures, and the TRA/podocalyxin combination formed therefrom. Podocalyxin-specific and TRA-specific antibodies, such as Mab84, TRA-1-60 and/or TRA-1-81 may be used. The TRA-1-60 and/or TRA-1-81 antibody is humanized for use in a human patient. Without intending to be bound by any particular theory, it is believed that these antibodies, such as the TRA-, Mab-84- and podocalyxin-specific antibodies, e.g., 3D3, can serve as a drug or biomolecule delivery system within the body of the patient.

In an aspect, the invention provides a system or device for detecting the presence or absence of podocalyxin and/or TRA molecules, e.g., biomarkers, in a blood sample and a diagnosis of the presence or absence of circulating tumor cells in a patient. The device can include a tool for obtaining the blood sample from the patient. Suitable tools are well known in the art and can include conventional syringes. The device further includes a means or mechanism for detecting podocalyxin and/or TRA molecules and determining their presence or absence in the sample. In certain embodiments, a detector means is employed to determine the presence or absence of anti-podocalyxin, TRA-1-60 and/or TRA-1-81 in the specimen. Furthermore, the device includes an indicator means or mechanism for converting and/or transmitting the result of the detector means, e.g., the presence or absence of anti-podocalyxin, TRA-1-60 and/or TRA-1-81 in the sample, to a displayable output to be read and/or interpreted by the patient and/or a medical professional.

In certain embodiments, the system or device includes a kit, e.g., test kit, for obtaining a blood sample from a patient, evaluating the blood sample to determine the presence of circulating tumor cells, and providing a diagnosis of aggressive or metastatic cancer in the patient. The kit can be employed by the patient to perform self testing and diagnosis or by a third party, e.g., a physician, to perform testing and diagnosis on another person. In certain embodiments, the kit may include a test strip, or similar configuration, having at least one region for testing for aggressive or metastatic cancer and may provide a visual indication, either with the naked eye or through a proper instrument, of whether there is the presence or absence of circulating tumor cells and, therefore, whether there is a positive or negative diagnosis of aggressive or metastatic cancer. An indication as to the presence of circulating tumor cells and an indication as to the presence of aggressive or metastatic cancer may be provided in the same or in different regions on the test strip. It is contemplated that a sufficient change in the region(s) of the test strip will provide the presence of circulating tumor cells and aggressive or metastatic cancer. It is further contemplated that the test strip may be immersed in the blood sample or a drop or portion of the sample may be applied to or deposited on the test strip. An adequate period of time may be needed to allow for a reaction to occur; e.g., a few seconds or a few minutes. In certain embodiments, the test strip region(s) involve a reaction with TRA-1-60 and/or TRA-1-81 monoclonal antibodies. The region(s) of the test strip can be compared with a chart, e.g., a color chart, to determine the presence, absence or level of circulating tumor cells in the sample. The comparison can be made by the naked eye or alternatively, can be read by an instrument. Further, the instrument may be connected to a display mechanism.

The number of cells in a blood sample that express TRA-1-60 , TRA-1-81 or podocalyxin may be measured by Fluorescence-activated cell sorting (FACS) methodology or by any other methodology that separates blood cells using antibodies or other molecules that bind to and interact with TRA-1-60, TRA-1-81, or podocalyxin. A kit may be developed which could be used with a FACS machine or with any other equipment that can sort and separated or count cells within a blood sample.

It will be appreciated that kits in accordance with the invention may include alternate designs and configurations.

In one embodiment, this invention is used for the detection and diagnosis of aggressive prostate cancer in male patients. In other embodiments, this invention is used for the detection and diagnosis of other aggressive cancers in both male and female patients.

It is understood and contemplated that in addition to or in place of podocalyxin and/or TRA positive circulating tumor cells, exosomes may be present in the peripheral whole blood of a patient and therefore, in a blood sample obtained from the patient. The exosomes may be positive for TRA and/or podocalyxin. Exosomes, which may be referred to as microvesicles in the art, are small lipid vesicles composed of cell plasma membrane and are secreted by normal and cancer cells by the process of exocytosis. The exosomes can express proteins and carbohydrate molecules on their surface, such as podocalyxin and TRA. Thus, the presence of podocalyxin- and/or TRA-expressing exosomes in a blood sample of the patient would be a positive marker for the presence of metastatic or aggressive cancer stem cells in the patient, as well as a measure of how aggressive is a local primary tumor.

### EXAMPLE

Two tubes of 8 milliliters of peripheral blood from a patient with metastatic breast cancer was obtained from a commercial supplier (Conversant Healthcare, Huntsville, Ala.) with Institutional Review Board consent. The blood was collected into EDTA test tubes (Vacutainer, BD, Franklin Lakes, N.J.) and was processed on the same day it was collected. The peripheral blood mononuclear cell fraction by Ficoll-Paque plus (GE Healthcare, Pittsburgh, Pa.) separation and the mononuclear cell fraction was resuspended in 1 ml of binding buffer (CTC Enrichment Kit, Fluxion Biosciences Inc., South San Francisco, Calif.). The mononuclear cell fraction was incubated with 4.5 microcon immunomagnetic beads which were coated with monoclonal anti-mouse Fc antibody and Bstrongomab 9A monoclonal antibodies. Bstrongomab 9A is a mouse monoclonal antibody developed by CureMeta LLC (Boston, Mass.) that binds to the TRA-1-60 and TRA-1-81 specific carbohydrate structure that is present on embryonic stem cells and on pluripotent cancer stem cells. The cells and beads were incubated for 2 hours at 4° C. with passive mixing. Bstrongomab9A positive blood cells were isolated with a IsoFlux System (Fluxion Biosciences Inc., South San Francisco, Calif.): The cell/bead suspension was loaded onto the microfluidic cartridge and processed at a flow rate of 20 microliters per minute. Isolated cells were recovered into a microfuge tube for further studies.

To determine the presence of TRA-1-60 or TRA-1-81 positive cells isolated on the Isoflux system we used the immunofluorescence microscopy technique. Isolated cells were fixed in phosphate-buffered saline buffer (PBS) containing 1.8% formaldehyde, washed with PBS, and blocked with 20% goat sera and 3% bovine serum albumin in PBS. The cells were stained for TRA-1-60 by incubating the fixed cells with mouse anti-TRA-1-60 monoclonal antibodies (Life Technologies, Grand Island, NY, 1:50 working dilution of primary antibody), or the cells were stained for TRA-1-81 by incubating the fixed cells with mouse anti-TRA-1-81 monoclonal antibodies (Life Technologies, Grand Island, NY, 1:50 working dilution of primary antibody). As a control, no primary antibodies were incubated with cells. Tra-1-60, TRA-1-81 and control cells were incubated with Alexa Fluor 488 goat anti-mouse IGM (Life Technologies, Grand Island, N.Y., at 1:250 working secondary antibody dilution) for 1 hour at room temperature followed by PBS washes to remove non-binding antibody. To identify positive TRA-1-60 or TRA-1-81 cells, the cells were loaded on poly-prep slides (Sigma, St. Louis, Mo.) and sealed with Prolong Gold antifade reagent with DAPI (Life Technologies, Grand Island, N.Y.) and light and fluorescence microscopy was done using an EVOS microscope (Advance Microscopy Group, Life Technologies, Grand Island, N.Y.).

The results of TRA-1-60 staining of mononuclear blood cells isolated with Bstrongomab 9A antibody demonstrated approximately 700 mononuclear blood cells were isolated from 8 milliliters of blood from a patient with metastatic breast cancer and 17 cells were identified as TRA-1-60 positive cells. These results showed that TRA-1-60 positive mononuclear cells with embryonic or pluripotent stem cell properties can be detected in a sample of blood from a patient with metastatic disease.

The results of TRA-1-81 staining of mononuclear blood cells isolated with Bstrongomab 9A antibody demonstrated that approximately 400 mononuclear blood cells were isolated from 4 milliliters of blood from a patient with metastatic breast cancer and 9 cells were identified as TRA-1-81 positive cells. These results showed that TRA-1-81 positive mononuclear cells with embryonic or pluripotent stem cell properties can be detected in a sample of blood from a patient with metastatic disease.

The results of staining with only secondary antibody of mononuclear blood cells isolated with Bstrongomab 9A antibody demonstrated that isolated cells from 4 milliliters of blood from a patient with metastatic breast cancer were stained only with Alexa Fluor 488 goat anti-mouse antibody and no positively stained cells could be detected in the sample. These results showed that secondary antibody alone did not stain the isolated blood cells and that TRA-1-60 or TRA-1-81 primary antibody was necessary to detect a positively stained cell in this assay.

Overall, these results showed that it is possible to use antibodies specific for the embryonic stem cell markers TRA-1-60 and TRA-1-81 to isolate and identify mononuclear blood cells that have embryonic or pluripotent stem cell properties in the blood of a patient with advanced and metastatic cancer.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

1. A method of detecting the presence of circulating tumor cells in a first patient, comprising:
obtaining a first blood sample from the first patient;
analyzing the first blood sample to determine a presence of cells that express podocalyxin and/or TRA molecules;
determining the presence of circulating tumor cells based on the presence of cells that express podocalyxin and/or TRA molecules in the first blood sample; and
determining the absence of circulating tumor cells based on the absence of cells that express podocalyxin and/or TRA molecules in the first blood sample.

2. The method of claim 1, further comprising providing a medical diagnosis of the presence of aggressive or metastatic cancer in the first patient based on the presence of the circulating tumor cells in the first blood sample, and providing a medical diagnosis of the absence of aggressive or metastatic cancer in the first patient based on the absence of the circulating tumor cells in the first blood sample.

3. The method of claim 1, further comprising comparing a number of cells that express podocalyxin and/or TRA in the first blood sample obtained from the first patient to a second blood sample selected from the group consisting of a control sample obtained from a second patient without cancer and an earlier obtained sample from the first patient.

4. The method of claim 3, wherein if the number of cells that express podocalyxin and/or TRA in the first blood sample is measurably higher than the number of cells that express podocalyxin and/or TRA in the second blood sample, providing a diagnosis of the presence of circulating tumor cells in the first patient.

5. The method of claim 3, wherein if the number of cells that express podocalyxin and/or TRA in the first blood sample is the same or less than the number of cells that express podocalyxin and/or TRA in the second blood sample, providing a diagnosis of the absence of circulating tumor cells in the first patient.

6. The method of claim 1, further comprising comparing a number of cells that express podocalyxin and/or TRA in the first blood sample obtained from the first patient to a threshold number of cells that express podocalyxin and/or TRA.

7. The method of claim 6, wherein if the number of cells that express podocalyxin and/or TRA in the first blood sample is measurably higher than or essentially equal to the threshold number of cells that express podocalyxin and/or TRA, providing a diagnosis of the presence of circulating tumor cells in the first patient.

8. The method of claim 6, wherein if the number of cells that express podocalyxin and/or TRA in the first blood sample is measurably less than the threshold number of cells that express podocalyxin and/or TRA, providing a diagnosis of the absence of circulating tumor cells in the patient.

9. The method of claim 1, further comprising :
providing a treatment regimen selected from the group consisting of radiation, chemotherapy, surgery and a combination thereof, for the presence of cells that express podocalyxin and/or TRA molecules in the first blood sample; and
providing a treatment regimen selected from the group consisting of active surveillance and monitoring for the absence of cells that express podocalyxin and/or TRA molecules in the first blood sample.

10. The method of claim 1, wherein the cancer is prostate cancer.

11. A system for detecting circulating tumor cells in a patient, comprising:
a tool for obtaining a blood sample from the patient;
a detector means for determining a presence or an absence of cells that express podocalyxin and/or TRA molecules in the blood sample; and
an indicator means to display the presence of circulating tumor cells based on the presence of cells that express podocalyxin and/or TRA molecules in the sample or to display the absence of circulating tumor cells based on the absence of cells that express podocalyxin and/or TRA molecules in the sample.

12. The system of claim 11, wherein said device is a test kit.

13. A method of detecting and diagnosing circulating tumor cells in a patient, comprising:
obtaining a blood sample from the patient;
analyzing the blood sample for a presence of cells that express podocalyxin and/or TRA molecules;
determining the presence or an absence of the cells that express podocalyxin and/or TRA molecules in the blood sample;
providing a diagnosis of the presence of circulating tumor cells in the patient based on the presence of cells that express podocalyxin and/or TRA molecules in the blood sample; and
providing a diagnosis of the absence of circulating tumor cells in the patient based on the absence of cells that express podocalyxin and/or TRA molecules in the blood sample.

14. The method of claim 13, wherein the analyzing of the blood sample comprises employing a binding material selected to interact with the podocalyxin and/or TRA molecules in the blood sample.
